SEQUENCE LISTING (14 sequences)

SEQ ID NO: 1 - DNA, 1437 bp, Anopheles gambiae
  source 1..1437: mol_type = genomic DNA
atgcaagtccagccgaccaagtacgtcggcctcgttgccgacctgatgccgaacattcggctgatgcaggccagcggtcactttctgttccgctacgtcaccggcccgatactgatccgcaaggtgtactcctggtggacgctcgccatggtgctgatccagttcttcgccatcctcggcaacctggcgacgaacgcggacgacgtgaacgagctgaccgccaacacgatcacgaccctgttcttcacgcactcggtcaccaagttcatctactttgcggtcaactcggagaacttctaccggacgctcgccatctggaaccagaccaacacgcacccgctgtttgccgaatcggacgcccggtaccattcgattgcgctcgccaagatgcggaagctgctggtgctggtgatggccaccaccgtcctgtcggttgtcgcctgggttacgataacatttttcggcgagagcgtcaagactgtgctcgataaggcaaccaacgagacgtacacggtggatataccccggctgcccatcaagtcctggtatccgtggaatgcaatgagcggaccggcgtacattttctctttcatctaccagatttacttcctgctgttttcgatggtccagagcaacctcgcggatgtcatgttctgctcctggttgctgctagcctgcgagcagctgcaacacttgaagggtattatgcgatcgttgatggagctttcggcctcgctggacacctaccggcccaactcttcgcaactgttccgagcaatttcagccggttccaaatcggagctgatcatcaacgaagaaaaggatccggacgttaaggactttgatctgagcggcatctacagctcgaaggcggactggggcgcccagttccgtgcgccgtcgacgctgcaaacgttcgacgagaatggcaggaacggaaatccgaacgggcttacccggaagcaggaaatgatggtgcgcagcgccatcaagtactgggtcgagcggcacaagcacgttgtacgtctcgtttcagcaatcggagatacgtacggtcctgccctgctgctacacatgctgacctccaccatcaagctgacgctgctcgcctaccaggcaacgaaaatcgacggtgtcaacgtgtacggattgaccgtaatcggatatttgtgctacgcgttggctcaggttttcctgttttgcatctttggcaatcggctcatcgaggagagctcatccgtgatggaggcggcctattcctgccactggtacgacgggtccgaggaggcaaaaaccttcgtccagatcgtttgtcagcagtgccagaaggcgatgactatttccggagccaagtttttcaccgtttcgctcgatctgtttgcttcggttcttggagccgttgtcacctacttcatggtgctggtgcagctgaagtaa

SEQ ID NO: 2 - protein, 478 aa, Anopheles gambiae
MQVQPTKYVGLVADLMPNIRLMQASGHFLFRYVTGPILIRKVYSWWTLAMVLIQFFAILGNLATNADDVNELTANTITTLFFTHSVTKFIYFAVNSENFYRTLAIWNQTNTHPLFAESDARYHSIALAKMRKLLVLVMATTVLSVVAWVTITFFGESVKTVLDKATNETYTVDIPRLPIKSWYPWNAMSGPAYIFSFIYQIYFLLFSMVQSNLADVMFCSWLLLACEQLQHLKGIMRSLMELSASLDTYRPNSSQLFRAISAGSKSELIINEEKDPDVKDFDLSGIYSSKADWGAQFRAPSTLQTFDENGRNGNPNGLTRKQEMMVRSAIKYWVERHKHVVRLVSAIGDTYGPALLLHMLTSTIKLTLLAYQATKIDGVNVYGLTVIGYLCYALAQVFLFCIFGNRLIEESSSVMEAAYSCHWYDGSEEAKTFVQIVCQQCQKAMTISGAKFFTVSLDLFASVLGAVVTYFMVLVQLK

SEQ ID NO: 3 - DNA, 1437 bp, Aedes aegypti
  source 1..1437: mol_type = genomic DNA
atgaacgtccaaccgacaaaataccatgggctggtgctcgacttgatgccgaacattcggctgatgcaaggcttcggtcactttcttttccgttacgtaaatgggccggtcctgattcggaagctgtactcctggtggaacctgataatgatcctgctgcagtattttgccatcatgggcaacctggtgatgaatacaggggacgtcaatgagctaacggcaaacaccataacgacgctgtttttcacccattccgtgaccaagttcatctacgtcgccgtcaactcggaacatttctaccgcacgctgggcatctggaatcaaccgaacagtcattcactttttgccgaatcggatgctcggtaccattcgattgcgttggctaagatgcgaaaactgctggtcatggtgatggtgactacagtgctatccgtcgtcgcatggatcacgataacattcttcggcgatagcgtcaaaaatgtattcgacaaagagactaatgaaacgtatacggtggaaattccccgattgcccatcaaggcttggtacccgtgggatgcaatgagcggagtgccgtactttttctccttcatctaccaggcttatttcctgctgttttcgatgtgccaggccaacctcgccgatgtgatgttctgctcctggctgctcttcacttgcgaacagctgcagcatttgaagggtataatgcgccccctgatggaactttccgccacgctggacacctaccgaccaaactcggctgccctgttccgtgtcgcttccgccggatccaaatcggagctgattttgaatgaagagaaagatcccgacacgaaagatttcgacttgaacggtatctacaactcgaaagcggactggggtgcacagttcagggcgccatccactttgcaaacgttcggtgacaatggtatcaatggtaatccaaatggactaaccaagaagcaggaactgatggtccgaagtgcgatcaagtactgggtggagaggcacaagcacgtcgttcgcctcgtatcggccatcggtgaaacttacggagccgccctgttgcttcacatgttgacctcgaccatcaagctgaccctgttggcttaccaggcaaccaaaatcgatgcactcaacgtttatggactgaccgtgatcggctatctggtctatgctctggctcaggtgttcctgttttgcattttcggcaatcgattaattgaagagagttcatcagtgatggaggctgcctactcgtgccactggtatgacggttccgaggaagccaaaactttcgtgcaaatcgtttgccagcagtgtcagaaagcgatgaccatatccggggccaagtttttcaccgtttcactggatctgttcgcatcggttctgggagcggtcgtcacctacttcatggtgttggtgcagttgaaataa

SEQ ID NO: 4 - protein, 478 aa, Aedes aegypti
MNVQPTKYHGLVLDLMPNIRLMQGFGHFLFRYVNGPVLIRKLYSWWNLIMILLQYFAIMGNLVMNTGDVNELTANTITTLFFTHSVTKFIYVAVNSEHFYRTLGIWNQPNSHSLFAESDARYHSIALAKMRKLLVMVMVTTVLSVVAWITITFFGDSVKNVFDKETNETYTVEIPRLPIKAWYPWDAMSGVPYFFSFIYQAYFLLFSMCQANLADVMFCSWLLFTCEQLQHLKGIMRPLMELSATLDTYRPNSAALFRVASAGSKSELILNEEKDPDTKDFDLNGIYNSKADWGAQFRAPSTLQTFGDNGINGNPNGLTKKQELMVRSAIKYWVERHKHVVRLVSAIGETYGAALLLHMLTSTIKLTLLAYQATKIDALNVYGLTVIGYLVYALAQVFLFCIFGNRLIEESSSVMEAAYSCHWYDGSEEAKTFVQIVCQQCQKAMTISGAKFFTVSLDLFASVLGAVVTYFMVLVQLK

SEQ ID NO: 5 - DNA, 1131 bp, Aedes aegypti
  source 1..1131: mol_type = genomic DNA
atgttgatagaaaattgtccaatcatcaacgtcaacgtcaaagtgtggctcttctgggcatatctccggaaacccaagtggtacagttatctgttgggatgcgttccggtgacggtgctgaacgtgttccaatttatgaacctttttcacgtgattgcgtctggcagcggcgatatgaacaagattatcatcgacgggtactttacggtgctctacttcaatttggtgcttcggacatccttcctaatggggaaccgaggcaagttcgaaacgtttctggagggaattgctgatgagtacgccgttctggagaagcaaaatgacatccgcccactattggatcagttgacccgtcgagcaaggattctgtccaaatcgaatctttggctgggagcgtttatcagtgcctgcttcgttacgtatcctctgttttcgccggacagtggccttccctacggagtttatattcccggggtcgacgtccacgcgtcgccaatttacgaaattgtgttcgtgctacagatttatctcacctttccggcatgctgcatgtacatcccattctctagcttctactgcacctgtgccctgttcggattggtacgaatcgcagcactgaaacggtccctggagaaaatccacgagtacaatacttccccccgatcgttatttgcgaggataaaagagtgtcttcagtatcacaaggacataatcaaatatgtgagcgaccttaacgaactggtgacctatatatttctactggagctgctttcgttcgggatgatgctgtgcgccttgctcttcctgttgagcatcagcaatcagttggcccagatggtaatgattggttcgtacatcttcatgattctgtcgcaaatgtacgccctctattggcactcgaatgaggtacgggaacagagcttggagattggtgactctctgtattataacagtgcttggctcgattttgacaactcggtgaaaaagaagattatcttgatgcttgcacgggcgcaacggccattagcgataaaaataggaaacgtctacccaatgacactggaaatgtttcaatcgttgctaaatgcgtcgtattcgtattttacattgctccgcagggtttacaattga

SEQ ID NO: 6 - protein, 376 aa, Aedes aegypti
MLIENCPIINVNVKVWLFWAYLRKPKWYSYLLGCVPVTVLNVFQFMNLFHVIASGSGDMNKIIIDGYFTVLYFNLVLRTSFLMGNRGKFETFLEGIADEYAVLEKQNDIRPLLDQLTRRARILSKSNLWLGAFISACFVTYPLFSPDSGLPYGVYIPGVDVHASPIYEIVFVLQIYLTFPACCMYIPFSSFYCTCALFGLVRIAALKRSLEKIHEYNTSPRSLFARIKECLQYHKDIIKYVSDLNELVTYIFLLELLSFGMMLCALLFLLSISNQLAQMVMIGSYIFMILSQMYALYWHSNEVREQSLEIGDSLYYNSAWLDFDNSVKKKIILMLARAQRPLAIKIGNVYPMTLEMFQSLLNASYSYFTLLRRVYN

SEQ ID NO: 7 - DNA, 1380 bp, Aedes aegypti
  source 1..1380: mol_type = genomic DNA
atgaaaaaggaagaaatttttaaggaaatccatcaggagctatccattgacctaaaaaatttacaacgttttacaatttttcttcttctaaccatttcgcgaaatgctctcatgggaggtaagttctcaataaatttgcgaattgttcaacccactcggcttattcttgatccatcccttctcttagctttcaaacgccgacaaatgaacgacctggtgaagttcgagtccttcattcgggtgccggagatattcttcgacatgatcggaatcacacgctacggggaagctcgagatacctggaaggctcgactcaagcaggcgttcttctggagttcatacgccaacacaattttctgtctgatcattgagcatatctacttcatcaaagcggcggggaacttcacgaactttttggaattgacggccttggcaccgtgcatcggattcacggcgctgtcgatagtcaaaattatgacgatcaagttgaacgaagccaagttgaacggaattctcgatcggcttagtgacttattcccgaggagtcacctcgatcaggatcgatatcgaacgtacaactataatctcgagtcgcaaatggtgatgaagtcattctcgattctctacatgatcttgatttggatcttcaatctgcttccgttggtatcgatgttggtgaactacatttcgactggaatactggaaaaggagcttccctacttcatgtggtattggtatgattggcacaaggcaggttactacgagataacgttcttccaccagaactggggagccttcgattcagctgtgttcaacctcagcacggatttactcttttgtgcaatcattcttctgatttgtttgcaatttgacatattggcgtacagattgcgacatgctaaaggtgattataaggagttggaacaatgtgtaaaactgcatcagtcagtcgttgagttgagcaaccagctggaaggaatattctcaccttccattttggtcaactttgtcggaagctcggtaataatctgtctggtgggattccaagctacgtccaatatcagtgcattcgatttgttcaaattcattctctttctgatttcatcgttagttcaagtttttctattgtgctactacggaaacaaattgattgaagcgagttcacaaatagggtactgtgccttcgaaggtacatggtacatggcagacttgcgctaccaaaagtcgcttcttttcgtgatgactcgagctggccagtggcaaaagctgacggcgatgaaattctccgtagtttcgttggcaagctattcggcgatcttaagtacgtcgttttcatacttcaccttgctgaagaccatctatgaaccaagtcagaagtga

SEQ ID NO: 8 - protein, 459 aa, Aedes aegypti
MKKEEIFKEIHQELSIDLKNLQRFTIFLLLTISRNALMGGKFSINLRIVQPTRLILDPSLLLAFKRRQMNDLVKFESFIRVPEIFFDMIGITRYGEARDTWKARLKQAFFWSSYANTIFCLIIEHIYFIKAAGNFTNFLELTALAPCIGFTALSIVKIMTIKLNEAKLNGILDRLSDLFPRSHLDQDRYRTYNYNLESQMVMKSFSILYMILIWIFNLLPLVSMLVNYISTGILEKELPYFMWYWYDWHKAGYYEITFFHQNWGAFDSAVFNLSTDLLFCAIILLICLQFDILAYRLRHAKGDYKELEQCVKLHQSVVELSNQLEGIFSPSILVNFVGSSVIICLVGFQATSNISAFDLFKFILFLISSLVQVFLLCYYGNKLIEASSQIGYCAFEGTWYMADLRYQKSLLFVMTRAGQWQKLTAMKFSVVSLASYSAILSTSFSYFTLLKTIYEPSQK

SEQ ID NO: 9 - DNA, 1263 bp, Aedes aegypti
  source 1..1263: mol_type = genomic DNA
atgcagctgaaagacgaatggatccaggaggacgacgtttacgacaatccgttgctccggttgaccatcaacgggttgaaatactatgggattttgctctataagagccaaccgtttaagaagttgaactgctttaggggagtttgcttcacggcgtccatgctggcgtttaacgttacgcagtatgtcgatctctatcaggtgtggggaaacattgccgaaatgacggccaatgcggccactacgcttctttttacaacaacaatcgtgagaattctgcatttctactggaatcgtgctaggttcaacaacgctataaaagtcgctgacgaaggtgtccagcatctgcttcgatttggaaatgctccggagaaggaaattttctgggacaatgttaaatacatgaatcgtttgactgcagcattttggatttgtgcattagttacggccaataccatgtgtgtttacgcgttggttcaatatcaatccctgaaatccatggattcgttcaactcaacggaacccttcgacccgcccacgattttgcgttcttggtatccaacggacaacatcgtggacagctttgccaccatttatctcatacagttgtacatcatgtatgtgggtcagctaattgttccatgttggcatgtttttatggttagcttgatgctgtacgctcgaactgcgttgatggccctgaactacaagttggctaacctggaacagtatgcagtgtcgggaatgcgtagcggacggaaaatcaagtgcgtcgtagatcctgaagaggaacgatgtaacgttcggaaggagctgattgtggaatgcattcaacagcagcacaagatcttcgagtacactcgggaattggaagccttgacaaggggtgccatgtttatggactttgtggtattttctgtgttactttgcgcactgctgtttgaagcttcttcgacaaactcatttgtgcaaatatttatagacatttgctatattatgaccatgaccgctatgctgttcctctattattggcacgccaacgagatacactatcaggccaaccttctttccagctcggcattcatgaacgactggtataactatccccgctcggttaaccgccatttgatcaccttcatctgctacagcaacaaaccgcttgacatgaaagcctacattgtttcgatgtcactggatactttcctggcgattctccgagcttcttacagctatttcacgatcctgaaacaagctgccggctaa

SEQ ID NO: 10 - protein, 420 aa, Aedes aegypti
MQLKDEWIQEDDVYDNPLLRLTINGLKYYGILLYKSQPFKKLNCFRGVCFTASMLAFNVTQYVDLYQVWGNIAEMTANAATTLLFTTTIVRILHFYWNRARFNNAIKVADEGVQHLLRFGNAPEKEIFWDNVKYMNRLTAAFWICALVTANTMCVYALVQYQSLKSMDSFNSTEPFDPPTILRSWYPTDNIVDSFATIYLIQLYIMYVGQLIVPCWHVFMVSLMLYARTALMALNYKLANLEQYAVSGMRSGRKIKCVVDPEEERCNVRKELIVECIQQQHKIFEYTRELEALTRGAMFMDFVVFSVLLCALLFEASSTNSFVQIFIDICYIMTMTAMLFLYYWHANEIHYQANLLSSSAFMNDWYNYPRSVNRHLITFICYSNKPLDMKAYIVSMSLDTFLAILRASYSYFTILKQAAG

SEQ ID NO: 11 - DNA, 1137 bp, Anopheles gambiae
  source 1..1137: mol_type = genomic DNA
atgctgatcgaagagtgtccgataattggtgtcaatgtgcgagtgtggctgttctggtcgtatctgcggcggccgcggttgtcccgctttctggtcggctgcatcccggtcgccgtgctgaacgttttccagttcctgaagctgtactcgtcctggggcgacatgagcgagctcatcatcaacggatactttaccgtgctgtactttaacctcgtcctccgaacctcctttctcgtgatcaatcgacggaaatttgagacattttttgaaggcgttgccgccgagtacgctctcctcgagaaaaatgacgacatccgacccgtgctggagcggtacacacggcggggacgcatgctatcgatatcgaatctgtggctcggcgccttcattagtgcctgctttgtgacctatcctctgtttgtgcccgggcgcggcctaccgtacggcgtcacgataccgggcgtggacgtgctggccaccccgacctaccaggtcgtgtttgtgctgcaggtttaccttaccttccccgcctgctgcatgtacatcccgttcaccagcttctacgcgacctgcacgctgtttgcgctcgtccagatagcggccctaaagcaacggctcggacgcttggggcgccacagcggcacgatggcttcgaccggacacagcgccggcacactgttcgccgagctgaaggagtgtctaaagtatcacaaacaaatcatccaatatgttcatgatctcaactcactcgtcacccatctgtgtctgctggagttcctgtcgttcgggatgatgctgtgcgcactgctgtttctgctaagcattagcaatcagctggcacagatgataatgattggatcgtacatcttcatgatactctcgcagatgtttgccttctattggcatgcgaacgaggtactggagcagagcctaggcattggcgatgccatttacaatggagcgtggccggactttgaggaaccgataaggaaacggttgattctaattattgcacgtgctcagcgaccgatggtgattaaagtcggcaacgtgtacccgatgacgttggaaatgtttcaaaaattgctcaacgtgtcctactcctatttcacactgctgcgccgagtgtacaactaa

SEQ ID NO: 12 - protein, 378 aa, Anopheles gambiae
MLIEECPIIGVNVRVWLFWSYLRRPRLSRFLVGCIPVAVLNVFQFLKLYSSWGDMSELIINGYFTVLYFNLVLRTSFLVINRRKFETFFEGVAAEYALLEKNDDIRPVLERYTRRGRMLSISNLWLGAFISACFVTYPLFVPGRGLPYGVTIPGVDVLATPTYQVVFVLQVYLTFPACCMYIPFTSFYATCTLFALVQIAALKQRLGRLGRHSGTMASTGHSAGTLFAELKECLKYHKQIIQYVHDLNSLVTHLCLLEFLSFGMMLCALLFLLSISNQLAQMIMIGSYIFMILSQMFAFYWHANEVLEQSLGIGDAIYNGAWPDFEEPIRKRLILIIARAQRPMVIKVGNVYPMTLEMFQKLLNVSYSYFTLLRRVYN

SEQ ID NO: 13 - DNA, 1200 bp, Anopheles gambiae
  source 1..1200: mol_type = genomic DNA
atggacccaccaacggacgagctggtgcgtttcgagtcgtttatccgtgtgccggaaattttcttcgccatgatcggtgtggcacggtacggtgagccgaagcgaacgcttcgggcgtacctgaagcatctgctcttctggtcgtcgtgcatcaataccggcttctgtctcgtgatcgagcatatctacttcgtgaaggcggccggtaactttaccaactttctgcagctgaccgcactggcaccgtgcatgggctttaccgcgctgtcgttcgtcaagatcatgacgatcaagctgaacgagacgaagctgaccgatatgctgcaccggctggatgcgctcttcccgagcacggtcgcactgcagcagcggtacggtgtgtaccagtacaaccgtgaatcgcaggtcgtgatgaaatcgttctcgatcctgtacatgatcctgatctggatgtttaacctgctgccgctggtgtcgatggtggcgggatacgttagcgacggcacgtggcacaagcagctgccgtactttatgtggtactggtacgattggcaccggcccggctacttcgcggtgacgtttctgcaccagaactggggcgggttcgtttcggccgtgttctacctctcgaccgatctgatgttctgtgcgatcgtgctgctgctctgtctgcagtttgacatcgtggcctaccggttgagccacgcgctgcccgatgaccaccaggagctggtcgggtgtgtgcgcatccatcaggcggtgatcgagctgtgcaatgagctggagcacatgttttccccctcgctgctggtcaactttctcagcagctccgtcatcatctgtttggtcgggtttcaggcgacggctgggattacgccggcggatctgttcaagtttgtgctgtttctcgtctcgtcgctggtgcaggtctttctgctgtgttactatggtaacaagctcatcgttgcgagctcgcaaattccctacagcgctttcgaggggaactggatcggggcgtccgtttcttaccagcgatcgctgctgttcgtgatgttacgctccaccaccgtgcagaagctgactgcgctgaaattctccatcgtttctctggcgagctattcgaagatactaagcacctcgttctcgtactttacgctactgaaagctatgtacgaacctaatgagaagaaaatgaagtga

SEQ ID NO: 14 - protein, 399 aa, Anopheles gambiae
MDPPTDELVRFESFIRVPEIFFAMIGVARYGEPKRTLRAYLKHLLFWSSCINTGFCLVIEHIYFVKAAGNFTNFLQLTALAPCMGFTALSFVKIMTIKLNETKLTDMLHRLDALFPSTVALQQRYGVYQYNRESQVVMKSFSILYMILIWMFNLLPLVSMVAGYVSDGTWHKQLPYFMWYWYDWHRPGYFAVTFLHQNWGGFVSAVFYLSTDLMFCAIVLLLCLQFDIVAYRLSHALPDDHQELVGCVRIHQAVIELCNELEHMFSPSLLVNFLSSSVIICLVGFQATAGITPADLFKFVLFLVSSLVQVFLLCYYGNKLIVASSQIPYSAFEGNWIGASVSYQRSLLFVMLRSTTVQKLTALKFSIVSLASYSKILSTSFSYFTLLKAMYEPNEKKMK

(19) United States
(12) Patent Application Publication
(10) Pub. No.: US 2025/0368699 A1
(43) Pub. Date: Dec. 4, 2025

(54) ARTHROPOD CHEMOSENSORY RECEPTORS AND USES THEREOF

(71) Applicant: Firmenich Incorporated, Plainsboro, CA (US)

(72) Inventors: Michael Saganich, San Diego, CA (US); Jose Mercado, Plainsboro, NJ (US); Mark Williams, San Diego, CA (US); Guy Servant, San Diego, CA (US); Min Lu Gallant, Plainsboro, NJ (US); Sebastian Chahda, Plainsboro, NJ (US)

(21) Appl. No.: 18/872,747

(22) PCT Filed: Jun. 7, 2023
(86) PCT No.: PCT/US2023/068085

§ 371 Date: Dec. 6, 2024

Related U.S. Application Data
(60) Provisional application No. 63/350,672, filed on Jun. 9, 2022.

(30) Foreign Application Priority Data
Jun. 21, 2022 (EP) 22180058.4

(51) Int. Cl. C07K 14/435; C12N 5/09; C12N 15/866; G01N 33/50; G01N 33/68

(52) U.S. Cl. CPC C07K 14/43577; C12N 5/0693; C12N 15/866; G01N 33/5008; G01N 33/68; C12N 2710/14043

(57) ABSTRACT

The present disclosure is directed to a mammalian cell comprising an arthropod chemosensory receptor and its uses in methods of identifying repellents and attractants or traps for use in an arthropod control article comprising an arthropod control composition. The cell may be contained in a cell-based assay or disposed on a solid support.

### REFERENCE TO THE SEQUENCE LISTING

The present application is being filed along with a Sequence Listing in electronic format. The Sequence Listing is provided as a file entitled SNMX.076PR.xml created on Jun. 6, 2023, which is 25,038 bytes in size. The information in the electronic format of the Sequence Listing is incorporated herein by reference in its entirety.

### INTRODUCTION

Many mammals, including humans, suffer from the action of arthropods. Some arthropods, such as for example mosquitoes and ticks, are not desirable for vertebrates such as mammals and in particular human subjects as they bite and, consequently, cause itching, transmission of diseases and/or germs or may be the cause for other diseases and/or conditions. Similarly, other pests indirectly affect human activity or society by eating, parasitizing, or destroying plant materials that are used as food, feed or raw materials. Still further pests are involved in the destruction or weakening of furniture or structures used or built by humans. These damages may directly be attributed to the arthropods or by their capability of spreading germs causing such issues.

Arthropod control compositions include active substances and when applied to skin, clothing, or other surfaces, they may discourage arthropods from landing or climbing on that surface. Arthropod control agents help preventing and controlling the outbreak of arthropod-borne diseases, such as malaria.

Arthropods use a combination of cues to find mammalians to prey upon. A primary means is by using chemosensory receptor to detect olfactory and taste cues in the environment. Chemosensory receptors are a large class of sensory receptors prevalent across the animal kingdom. In arthropods, odorant receptors (ORs) and ionotropic glutamate receptors (IRs) are two classes of ligand-gated ion channels and are commonly used to detect environmental cues.

With respect to mosquitoes, specific receptors are present in mosquito olfactory tissues. Mosquito odorant receptors (ORs) play key roles in mosquito olfactory behaviors and are composed of a highly conserved co-receptor, designated ORCO, and a conventional ligand-binding odorant receptors (ORX). The ORCO receptor is responsible for efficient expression of the ORX in the target tissue while the ORX confer odor selectivity. It is the expression profile, but most importantly, the function of these ORCO+ORs (ranging from a few to over a hundred within a single species) that determine a specific olfactory-mediated behavioral event.

The need of a reliable arthropod chemosensory receptor expression system is in high demand in order to screen a large number of compounds to identify the next generation of repellents and attractants/traps.

The present disclosure addresses this need by providing a reliable system for the expression, function and high-throughput screening of arthropod chemosensory receptor targets in mammalian cells.

### SUMMARY

A first aspect of the disclosure provides a mammalian cell comprising an arthropod chemosensory receptor.

An embodiment of this aspect of the disclosure is wherein the cell is from an immortalized cell line. Preferably the immortalized cell line is not an embryo-derived cell line. More preferably the cell is a U2OS cell.

A further embodiment of this aspect of the disclosure is wherein the receptor is transiently expressed in the mammalian cell. Preferably the receptor is transiently expressed using a baculovirus expression vector system.

A further embodiment of this aspect of the disclosure is wherein the chemosensory receptor is an olfactory receptor.

A further embodiment of this aspect of the disclosure is wherein the arthropod is an insect and the chemosensory receptor is an insect odorant receptor.

Preferably the mosquito odorant receptor comprises an odorant receptor (ORX) and a co-receptor (ORCO). Preferably the mosquito odorant receptor is an Aedes aegypti and/or Anopheles gambiae odorant receptor.

Further embodiments of this aspect of the disclosure is wherein the co-receptor (ORCO) is selected from AgamOrco and/or AaORCO and the receptor (ORX) is selected from AaOR8, AaOR2 or AaOR11, AgamOR8 or AgamOR2. Preferably the cell comprises AgamORCO and AgamOR8 or AgamOR2. Preferably the cell comprises AaORCO with AaOR8, AaOR2 or AaOR11.

A further embodiment of this aspect of the disclosure is wherein the cell is contained in a cell-based assay or disposed on a solid support.

A further aspect of the disclosure provides a method of identifying an arthropod control agent, the method comprising:
(a) contacting one or more U2OS cells of any one of the previous claims with a test compound; and
(b) measuring a receptor activity,
wherein a change in the receptor indicates that the test compound is an arthropod control agent modulator.

A further aspect of the disclosure provides the use of an identified compound of the aspects of the disclosure as an arthropod repellant.

A further aspect of the disclosure provides an arthropod, preferably insect, control article comprising an arthropod, preferably insect, control composition as defined in the disclosure,

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the present disclosure.

FIG. 1 U2OS cells infected with the odorant receptor co-subunit AaORCO provided a greater fluorescence assay window than HEK293 cell stably expressing the same gene.

FIG. 2A greater fluorescence assay window was observed in infected U2OS compared to infected HEK293 cells regardless of mosquito odorant receptor target or species expressed.

FIG. 3: Effective use of the Bacmam expression system and U2OS cells for high-throughput screening and identification insect olfactory receptor modulators.

FIG. 4 shows the successful use of U2OS cells on an automatic patch-clamp (APC) instrument to measure mosquito odorant receptor channel currents elicited by a ligand. In this example, U2OS cells were co-transduced with AaORCO and AaOR8 channel subunits overnight and stimulated with increasing concentrations of 1-octen-3-ol ligand in the absence and presence of 10 μM VUAA1. Dose-dependent activation of AaORCO+AaOR8 receptors was observed (EC50=537 nM) along with a left-shift in channel 1-octen-3-ol sensitivity in the presence of VUAA1 (EC50=101 nM). This example shows U2OS host cells express functional insect receptor ion channels at the plasma membrane and are amenable for use on high-throughput electrophysiology screening platforms for the discovery of novel ion-channel modulators for use as insect repellants and attractants.

### DETAILED DESCRIPTION

The following detailed description sets forth various aspects and embodiments provided herein. The description is to be read from the perspective of the person of ordinary skill in the relevant art. Therefore, information that is well known to such ordinarily skilled artisans is not necessarily included.

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary,

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A1 and A2, then one or more members of the class can be present concurrently.

The first aspect of the disclosure provides a mammalian cell comprising a arthropod chemosensory receptor.

By “mammalian cell” we include cells from any species of mammal, including but not limited to: rodents, in particular rats, mice, hamsters; leporidae including rabbits and hares; primates including humans, chimpanzees, bonobos, macaques including cynomolgus monkeys. A preferred embodiment is wherein the cell is a human cell.

In some instances, cells besides human U2OS cells can also be used. Non-limiting examples of such cells include the following cell types: 1A2, ARH-77, RWPE-1, WI-38, EJM, NCI-H1155, L-1236, NCI-H526, JM1, SHP-77, SNU-878, NCI-H2196, C3A, CA46, SNU-466, KS-1, SNU-738, MOLP-2, HDLM-2, Pfeiffer, HCC-15, Alexander cells, L-540, KMS-12-BM, JK-1, NCI-H1092, SW 1990, NCI-H1184, SU-DHL-1, Hep 3B2.1-7, P3HR-1, NCI-H2029, SU-DHL-5, SNU-1, MOLP-8, SUP-M2, MONO-MAC-1, SNU-1040, KYM-1, HEC-59, HCC1569, OCI-LY3, Hs 819.T, DU4475, CI-1, S-117, OVCAR-8, SNU-626, HL-60, SUIT-2, T3M-4, RKO, MOR/CPR, DK-MG, GA-10, OCUM-1, HCT-15, HT, MONO-MAC-6, G-402, Toledo, COV362, SU-DHL-8, Daoy, NCI-H1435, LS513, Hs 839.T, Hs 172.T, BT-483, KMS-21BM, AGS, NCI-H2172, LC-1/sq-SF, SNU-201, NUGC-4, SK-HEP-1, SUP-B15, SNU-5, HT-1197, SUP-T1, AMO-1, KU812, AN3 CA, AML-193, VMRC-RCW, HLE, HuH28, Hs 751.T, NCI-H2110, MEG-01, MV-4-11, Hep G2, KYSE-30, KALS-1, BICR 6, RMUG-S, JHH-6, Ki-JK, IST-MES1, HCC-95, HPB-ALL, HSC-3, 697, LOU-NH91, KARPAS-299, GI-1, COLO 792, SK-N-FI, D341 Med, HGC-27, SR-786, COLO-818, MHH-CALL-2, SF126, NCI-H322, A-253, NCI-H1623, MCF7, HCC-44, FU97, OCI-LY-19, Hs 766T, NCI-H522, RL, HCC1428, RPMI 6666, U-937, NCI-H460 , SW 1088, NCI-H1792, NCI-H1693, UACC-257, JHUEM-2, HuT 78, UACC-893, NCI-H929, A-704, OV56, LN-229, OE19, SK-MEL-24, RD-ES, NCI-H211, KCI-MOH1, NCI-H1963, Hs 706.T, ChaGo-K-1, EPLC-272H, OPM-2, KHM-1B, A549, HuG1-N, NCI-H508, MHH-CALL-3, SNU-1076, A3/KAW, MEL-HO, TO 175.T, Caki-1, Hs 936.T, SK-LU-1, WM-983B, K-562, EFE-184, SNU-520, NCI-H2291, HCC-1195, ABC-1, KE-39, NH-6, HCC2218, CMK, RS4;11, KYSE-450, OV7, KYSE-510, SK-UT-1, SNU-C1, OE33, P12-ICHIKAWA, DLD-1, COV434, HuNS1, SNU-899, SW480, COLO-678, LU99, KOPN-8, NCI-H2227, SW1463, Hs 675.T, JHH-4, NCI-H1703, HEC-1-A, BDCM, MIA PaCa-2, PC-3, TE-15, PK-45H, MKN-45, HCC-366, CAL-29, HEC-50B, CPC-N, KMRC-20, SW1116, EOL-1, COLO 205, EHEB, YD-38, MC116, SK-N-BE(2), BV-173, NCI-H2347, LU65, RT4, U-87 MG, LK-2, KP-N-YN, HEC-251, NCI-H1651, GP2d, RERF-LC-MS, NB-4, NCI-H2286, SNU-61, T-47D, huH-1, KYSE-180, ST486, SW 1353, M-07e, KASUMI-1, YH-13, NCI-H28, GAMG, JeKo-1, GOS-3, SNU-324, PA-TU-8902, MFE-280, SNU-245, NALM-1, RERF-LC-Sq1, BICR 22, ZR-75-1, COR-L23, SW579, COR-L88, KM12, Hs 611.T, OUMS-23, RERF-LC-Ad1, NCI-H1385, SK-LMS-1, COLO-320, BL-70, GRANTA-519, MCAS, Panc 08.13, AM-38, KMS-11, SIG-M5, SNU-407, JHOS-2, OVCAR-4, Set-2, OV-90, MeWo, HEL, HT-29, MDA-MB-231, TOV-21G, NCI-H1355, KMS-27, NALM-6, KMS-26, Caov-4, KASUMI-2, UACC-62, U266B1, Hs 695T, HT55, BICR 31, TCC-PAN2, KMS-20, Hs 578T, RI-1, Hs 606.T, NCI-H1341, THP-1, BCP-1, Hs 737.T, SW1417, MOLT-4, Raji, ESS-1, MEL-JUSO, SH-10-TC, Hs 683, ME-1, EB2, PLC/PRF/5, NCI-H1339, A4/Fuk, SEM, HEC-265, IST-MES2, KE-97, NCI-H1437, COLO-704, NCI-H1915, TE-5, NCI-H2023, NCI-H82, T1-73, SNU-840, HuT 102, NCI-H1944, KYSE-520, Kasumi-6, 1321N1, Hs 742.T, IM95, PL45, CL-40, WM1799, KMM-1, SNU-449, JHUEM-1, KARPAS-620, Loucy, SNU-1079, Daudi, HCC-56, HSC-2, COR-L47, PA-TU-89885, OAW28, COR-L311, L-363, Malme-3M, NOMO-1, Hs 870.T, SU-DHL-10, Hs 229.T, NCI-H810, KYSE-410, RPMI-8402, SNU-175, EBC-1, RVH-421, K029AX, PA-TU-8988T, LXF-289, OVSAHO, CAL-12T, Hs 940.T, MM1-S, SUP-HD1, LNCaP clone FGC, HSC-4, NU-DHL-1, NCI-H2228, BEN, CAL-78, Sq-1, NCI-H1793, SNU-C2A, MDA-MB-134-VI, COV318, KE-37, TYK-nu, MOTN-1, T98G, SW837, EB1, Becker, PE/CA-PJ34 (clone C12), Hs 616.T, NCI-H446, WM-88, CHP-126, Calu-1, SNU-283, NCI-H1573, SW 1271, SNU-16, JHOS-4, ACHN, Calu-3, KMRC-1, SW 1783, TE-11, TE-9, HuH-6, P31/FUJ, HT-1376, NCI-H520, 786-O, KNS-60, Caki-2, OVK18, PL-21, NCI-H2452, JURL-MK1, TEN, JHH-7, MDA-MB-157, Calu-6, RKN, NUGC-2, ONS-76, J82, OUMS-27, SNU-1196, Hs 739.T, RPMI-7951, NCI-H854, JHH-5, JVM-2, Hey-A8, 5637, KYSE-140, Capan-2, KYSE-150, HEC-1-B, BICR 16, HEL 92.1.7, MHH-NB-11, SNU-387, SK-OV-3, SK-MEL-28, IGROV1, ML-1, HLF-a, CHL-1, YKG1, A-204, OCI-M1, 8505C, JVM-3, NCI-H647, DB, COLO-800, PK-59, FaDu, HLF, OVMANA, EFO-27, PF-382, NCI-H747, LS123, SU-DHL-6, SJRH30, PANC-1, NCI-H2342, KM-H2, DND-41, HH, HuCCT1, F-36P, DMS 454, Hs 274.T, AU565, NCI-H1666, EN, RH-41, NCI-H1373, NCI-H838, SK-MEL-30, MOLM-6, DEL, NCI-H226, NCI-H1648, NCI-H661, 143B, Mino, C32, KMS-34, NCI-H1694, SK-ES-1, UACC-812, GDM-1, NCI-H23, Panc 02.03, CCF-STTG1, LOX IMVI, SJSA-1, MDST8, PK-1, NCI-H716, SU-DHL-4, MPP 89, MJ, COLO 829, PE/CA-PJ15, HD-MY-Z, BxPC-3, WM-793, COLO 668, T84, JHOM-1, PEER, LS411N, GMS-10, KMBC-2, RMG-I, KELLY, SNU-761, NALM-19, HEC-151, G-361, OVTOKO, A-498, SW 900, LCLC-103H, FTC-133, QGP-1, Reh, CMK-11-5, NU-DUL-1, BT-20, Hs 600.T, Hs 604.T, KATO III, SNU-410, NCI-H2126, SK-MEL-5, MDA-MB-468, AsPC-1, HUP-T3, KP-N-SI9s, L-428, SNU-1105, HUP-T4, 769-P, LMSU, NCI-H1869, NCO2, MOLM-16, CAL 27, HCC70, NCI-H1930, COV644, Hs 863.T, HCC-2279, D283 Med, Hs 944.T, HCC1599, MDA-MB-415, HCC2157, NCI-H1618, SNU-308, HCC1954, DMS 153, HPAF-II, T24, CJM, VM-CUB1, UM-UC-3, LAMA-84, NCI-H1734, JHH-2, VMRC-RCZ, MFE-319, MDA-MB-453, SNU-503, TOV-112D, B-CPAP, GSU, HCC-78, NCI-H2171, CAMA-1, HEC-108, HCC4006, CAL-85-1, NCI-H2122, COLO-699, NCI-H196, LUDLU-1, SW 780, RPMI 8226, LP-1, PC-14, HuTu 80, T.T, SW948, 22Rv1, HARA, NCI-H596, IPC-298, SCaBER, NCI-H1838, NB-1, Hs 934.T, Hs 895.T, DMS 114, KYSE-70, KP-3, KP4, DAN-G, NCI-H2009, OC 316, SCC-25, U-138 MG, RCC10RGB, MFE-296, NCI-H1755, RERF-LC-KJ, 8305C, WSU-DLCL2, ES-2, MSTO-211H, SCC-15, ZR-75-30, PSN1, SNU-423, NCI-H2106, TE-1, UT-7, KMS-28BM, NCI-H2081, SK-MM-2, COLO 741, OC 314, HCC1395, MOLT-13, LN-18, Panc 10.05, PE/CA-PJ41 (clone D2), Hs 746T, CW-2, SKM-1, NUGC-3, TE-10, NCI-H358, NCI-H69, BFTC-909, HOS, BICR 18, NCI-H1395, OVKATE, Hs 698.T, EFM-19, COLO-783, MHH-CALL-4, ACC-MESO-1, NCI-H1436, KP-N-RT-BM-1, SK-MEL-31, NCI-H1105, CAL-51, YD-15, NCI-H2085, NCI-H2444, HCC1187, Hs 939.T, CAL-120, SCC-9, TUHR14TKB, KMRC-2, KG-1-C, ECC10, CGTH-W-1, NCI-H841, C2BBe1, SUP-T11, RCH-ACV, CADO-ES1, JURKAT, 647-V, SK-MEL-2, MDA-MB-175-VII, MKN74, SNU-C4, LCLC-97TM1, SCC-4, BHY, IGR-37, KYO-1, Hs 281.T, TT, TUHR4TKB, HT-1080, NCI-H660, TE 441.T, LS1034, KNS-42, Panc 04.03, HCC1419, AZ-521, SNG-M, NCI-N87, G-292, clone A141B1, KPL-1, MDA-MB-361, CL-14, NCI-H2170, HuH-7, RD, NCI-H2066, IGR-1, TE-14, VCaP, BL-41, SNU-620, SK-MES-1, MEC-2, NCI-H1299 IGR-39, RT112/84, SF-295, DV-90, A2780, BICR 56, NCI-H510, NCI-H2141, YD-8, NCI-H2405, TF-1, MEC-1, CCK-81, NCI-H1048, Hs 822.T, NCI-H2052, K052, CAL-54, Hs 840.T, SW620, SK-CO-1, BT-474, CL-11, KNS-62, NCI-H1650, G-401, MOLT-16, SNU-398, COLO-680N, EM-2, Hs 294T, CAL-62, KMRC-3, A101D, KG-1, BT-549, HT115, A-375, SW-1710, WM-115, KLE, JHUEM-3, MKN7, CHP-212, HCC202, BC-3C, NCI-H1568, KMS-18, PE/CA-PJ49, COLO-849, SIMA, OCI-AML3, GSS, EC-GI-10, EFO-21, RCM-1, DMS 273, KU-19-19, RERF-GC-1B, SH-4, SK-MEL-3, RERF-LC-Ad2, M059K, JHOM-2B, MDA PCa 2b, Hs 852.T, RL95-2, Panc 03.27, SNU-216, Panc 02.13, CFPAC-1, SK-N-SH, OCI-AML2, LoVo, SBC-5, NCI-H1876, NCI-H441, SK-N-AS, COR-L24, HCC38, NCI-H1781, DOHH-2, NCI-H1563, U-251 MG, HPAC, JIMT-1, U-2 OS, A-673, TC-71, NCI-H650, NIH: OVCAR-3, CAS-1, JL-1, SK-MEL-1, MDA-MB-4355, Ishikawa (Heraklio) 02 ER-, TE 617.T, SU.86.86, RERF-LC-AI, TT2609-C02, LS 180, YAPC, HDQ-P1, KNS-81, FU-OV-1, KP-2, DMS 53, SNU-1272, Detroit 562, 42-MG-BA, L3.3, COLO-679, NCI-H2087, NCI-H2030, GCT, NCI-H889, Caov-3, MDA-MB-436, NCI-H524, MKN1, KCL-22, Capan-1, CML-T1, H4, NCI-H727, Hs 343.T, MHH-ES-1, NMC-G1, HCC-1171, REC-1, Hs 618.T, A172, YD-10B, SW48, MUTZ-5, TE-6, JHH-1, HCT 116, TE-4, IA-LM, MG-63, NCI-H1975, TALL-1, HCC1806, HMCB, SCLC-21H, HCC1500, CL-34, Panc 05.04, SW403, TM-31, HCC1937, JMSU-1, DMS 79, SNB-19, NCI-H1836, Li-7, HCC827, 639-V, MOLM-13, SK-BR-3, IMR-32, TUHR10TKB, OAW42, SK-N-MC, TGBC11TKB, NCI-H1581, EFM-192A, YMB-1, HCC2935, ECC12, HCC-33, DU 145, NCI-H146, SNU-1214, SNU-1077, 23132/87, HT-144, SNU-182, Hs 888.T, SNU-475, GCIY, Hs 729, JHOC-5, SW 1573, HEC-6, OCI-AMLS, Hs 688(A). T, Hs 821.T, PCM6, RT-112, SK-N-DZ, SNU-478, SNU-119, HCC1143, NCI-H209, 8-MG-BA, COR-L105, COR-L95, SNU-46, COV504, CAL-148, SNU-05, DBTRG-05 MG, BHT-101, WM-266-4, BFTC-905, KYSE-270, TE-8, SNU-213, and SH-SYSY.

A preferred embodiment of the disclosure is wherein the mammalian cell is an immortalized cell line.

An immortalised cell line is a population of cells from a multicellular organism which would normally not proliferate indefinitely but, due to mutation, have evaded normal cellular senescence and instead can keep undergoing division. The cells can therefore be grown for prolonged periods in vitro. The mutations required for immortality can occur naturally or be intentionally induced for experimental purposes. Immortal cell lines are a very important tool for research into the biochemistry and cell biology of multicellular organisms. Immortalised cell lines have many uses in biotechnology, in particular the use in screening assays where they have been modified to express cell receptor proteins.

Preferred immortalised cell lines of the disclosure include U2OS and HEK293 .

A particularly preferred embodiment of the disclosure is wherein the immortalized cell line is not an embryo-derived cell line. In particular it is preferred that the cell line is U2OS.

Arthropod chemosensory receptors show limited functional expression using common heterologous expression system and host cells such as HEK293 cells. Due to this difficulty, mosquito ORs are often studied using the Xenopus oocyte expression system. Unfortunately, the electrophysiological assay is very low throughput precluding the ability to screen thousands of compounds. A common strategy to overcome expression problems is the use of engineered stable cell lines. Another strategy is to mutate or add sequences to the gene to enhance receptor expression and function in mammalian cells. These strategies require considerable time and resources and, in the case of tagging and mutating the receptor, do not allow the expression and screening of the native (wild type) receptor. Most importantly, it precludes the systematic heterologous expression of the tens or hundreds of mosquito receptor genes and subunit combinations that can be found in a single mosquito species. An efficient and flexible expression system that enables the expression of mosquito odorant receptors in cells amenable to traditional high-throughput screening (HTS) methods is necessary.

The present disclosure, therefore, is directed to expression arthropod chemosensory receptors in different immortalized cell lines. Surprisingly, it was identified that the U2OS cell line allowed for functional expression in these cells.

Therefore, a preferred embodiment of the disclosure is wherein the mammalian cell is a U2OS cell.

Human U2OS cells are well known in the art and are generally available commercially, for example, from ATCC Catalog No. HTB-96.

An embodiment of this aspect of the disclosure is wherein the mosquito olfactory receptor is transiently expressed in the U2OS cell.

The term “transformation” or “to transform”, “transfection” or “to transfect” as used herein means any introduction of genetic material, into a mammalian host cell, wherein the mammalian host cell may be transiently transfected or stably transfected. The genetic material may be an expression vector comprising a gene of interest (e.g., a recombinant secreted therapeutic protein) or a polynucleotide sequence encoding siRNA or shRNA. It also means the introduction of a viral nucleic acid sequence in a way which is for the respective virus the naturally one. The viral nucleic acid sequence needs not to be present as a naked nucleic acid sequence but may be packaged in a viral protein envelope.

“Heterologous gene” or “heterologous sequences” can be introduced into a target cell directly (e.g., siRNAs) or by using an “expression vector”, preferably a mammalian expression vector. Methods used to construct vectors are well known to the person skilled in the art and described in various publications. In particular techniques for constructing suitable vectors, including a description of the functional components such as promoters, enhancers, termination and polyadenylation signals, selection markers, origins of replication, and splicing signals, are reviewed in considerable details in (Sambrook J, ef a/., 1989. Molecular Cloning: A Laboratory Manual. Cold Spring Harbor: Cold Spring Harbor Laboratory Press) and references cited therein. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses and bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operably linked, are well known in the art and some are commercially available from companies such as Stratagene, La Jolla, CA; Invitrogen, Carlsbad, CA; Promega, Madison, WI or BD Biosciences Clonetech, Palo Alto, CA. Usually expression vectors also comprise an expression cassette encoding a selectable marker, allowing selection of host cells carrying said expression marker.

Transfection of eukaryotic host cells with a polynucleotide or expression vector, resulting in genetically modified cells or transgenic cells, can be performed by any method known in the art (see e.g. Sambrook J, ef a/., 1989. Molecular Cloning: A Laboratory Manual. Cold Spring Harbor: Cold Spring Harbor Laboratory Press). Transfection methods include, but are not limited to liposome-mediated transfection, calcium phosphate co-precipitation, electroporation, nucleofection, nucleoporation, microporation, polycation (such as DEAE-dextran)-mediated transfection, protoplast fusion, viral infections and microinjection. The transformation may result in a transient or stable transformation of the host cells. Preferably, the transfection is a stable transfection. The transfection method that provides optimal transfection frequency and expression of the heterologous genes in the particular host cell line and type is favoured. Suitable methods can be determined by routine procedures. For stable transfectants the constructs are either integrated into the host cell's genome or an artificial chromosome/mini-chromosome or located episomally so as to be stably maintained within the host cell. Thus, the stably transfected sequences actually remain in the genome of the cell and its daughter cells. Typically, this involves the use of a selectable marker gene and the gene of interest or the polynucleotide sequence encoding the RNA is integrated together with the selectable marker gene. In some cases the entire expression vector integrates into the cell's genome, in other cases only parts of the expression vector integrate into the cell's genome. Cells “stably expressing” a recombinant secreted therapeutic protein or an RNA is stably transfected with a gene encoding said recombinant secreted therapeutic protein or with a polynucleotide sequence encoding said RNA. Thus, the sequences encoding the recombinant secreted therapeutic protein or RNA remain in the genome of the cell and its daughter cells.

An embodiment of this aspect of the disclosure is wherein the mosquito olfactory receptor is transiently expressed in the U2OS cell using a baculovirus expression vector system.

Baculovirus expression vector systems are well known in the art. The baculovirus expression vector (BEV) is a recombinant baculovirus with a double-stranded circular DNA genome that has been genetically modified to include a foreign gene of interest. BEVs are viable and can infect susceptible hosts. Therefore, BEVs can efficiently transfer foreign genes into these eukaryotic host cells. The foreign gene is usually a chimeric construct with the sequence encoding a protein of interest placed under the transcriptional control of a viral promoter. This arrangement enables viral functions to transcribe the gene during infection. The resulting mRNA is translated and the newly synthesized protein modified by host-encoded biosynthetic machinery.

Preferably the baculovirus expression vector system is a BacMam system. Baculovirus gene transfer into Mammalian cells, known from scientific research articles as BacMam, is the use of modified baculovirus to deliver genes to mammalian cells, wherein the modification is the inclusion of a mammalian recognizable promoter is located upstream of a gene to be expressed. BacMam expression vector systems are well known in the art and can be obtained from, for example, TheroFisher Scientific or prepared using the protocol outlined herein in the Examples section of this application.

The first aspect of the disclosure comprises a mammalian cell comprising an arthropod chemosensory receptor.

By “arthropod chemosensory receptor” we include any chemosensory receptor from any arthropod species.

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present disclosure's understanding relate to undesired arthropods, meaning that their presence in the air, on the surface of an article, the surface of a plant or the surface of a vertebrate, such as a human subject or other mammal, preferably human subject, is not desired. Preferably undesired arthropods are pest arthropods that impact plants and animals, e.g. thrips, aphids, beetles, moth, mealybug, scale etc., more preferably pest arthropods that impact animals, e.g. ants, termites, cockroaches, flies, etc., even more preferably blood feeding arthropods that impact vertebrates, e.g. biting fly, bed bug, kissing bug, flea, lice, mosquitos and ticks, even more preferably mosquitos and ticks.

The term “insect” has the normal understanding by a skilled person the technical field. An insect is described by a well-defined head, thorax, and abdomen, only three pairs of legs, and typically one or two pairs of wings

In a particular embodiment, the insect is a mosquito, biting fly, bedbug, kissing bug, flea, lice, ant, termite, cockroach, fly, aphid, beetle, thrips, moth, mealybug or scale bug, more preferably a mosquito.

The term “arachnid” has the normal understanding by a skilled person the technical field. An arachnid is described having a segmented body divided into two regions of which the anterior bears four pairs of legs but no antennae.

In a particular embodiment, the arachnid is a tick, mite, chigger or spider, more preferably a tick.

The term “chemosensory receptor” has the normal meaning for a skilled person in the technical field. Chemosensory receptors recognize chemical signals of odorants and tastants in the environment. Six multigene families are known to encode chemosensory receptors in vertebrates and four multigene families in insects, respectively. Near-complete repertoires of chemosensory receptor genes have been determined from the genomic sequences of various organisms. These studies have shown that the number of chemosensory receptor genes is generally large and varies greatly among different organisms. There are many publications disclosing the specific amino acid sequences of many of these receptor proteins. With respect to ticks (Ixodes scapularis) their genetic sequence is known and belong to the gustatory receptor and ionotropic glutamate receptor (iGluR)-related ionotropic receptor families.

A preferred embodiment of the disclosure is wherein the chemosensory receptor is an olfactory receptor. Olfactory receptors are a class of chemosensory receptors which are activated and stimulate a nerve, when the receptors is bound by a specific compound. Typically these compounds are volatile compounds, and can include gasses such as CO₂.

One class of olfactory receptor is the gustatory receptor (GRs). In insects, they there can be more than 50 GR genes present in a species, and are expressed in the labellum, legs, and pharynx of the adult fly, the larval taste organs, and in a variety of other adult tissues, including the antenna, maxillary palp, enteroendocrine cells of the gut, multidendritic cells of the abdominal body wall, neurons innervating reproductive organs, and the brain. In particular, studies revealed Gr21a and Gr63a are coexpressed as one class of antennal ORNs, where together they confer response to CO₂.

A further class of olfactory receptor is the ionotropic receptor (IRs). Ionotropic receptors are ligand-gated ion channels made up protein subunits that form an ion-conducting pore in the center of the receptor. There are four families of ionotropic receptors, which differ in their molecular structure and the ligands that open them. The effects of activating ionotropic receptors can be excitatory or inhibitory, according to the equilibrium potential for the ions they pass and the impact of these ion fluxes on membrane potential. In insects, particularly, Drosophila, around 17 IRs are expressed in the antenna, mostly in ORNs of coeloconic sensilla. These IRs confer response to many organic acids and amines. IR92a is required for response to particular amines, whereas IR64a is acid-sensitive. Like ORs, the trafficking and function of these IRs depend on the expression of widely expressed co-receptors, including IR8a or IR25a. For example, IR64a and IR8a are physically associated in vivo and form a functional channel when coexpressed in Xenopus oocytes. Some antennal IRs play interesting behavioral roles. IR84a is activated by food odors, and this activation increases levels of male courtship behavior. Evidently the ORN expressing IR84a influences a male courtship circuit. D. melanogaster mates primarily on food sources, and thus IR84a appears to provide a neural link between food and sex.

A preferred embodiment of the disclosure is wherein the receptor is a mosquito odorant receptor.

Mosquito odorant receptors (ORs) play key roles in mosquito olfactory behaviors and are composed of a highly conserved co-receptor, designated ORCO, and a conventional ligand-binding odorant receptors (ORX). The ORCO receptor is responsible for efficient expression of the ORX in the target tissue while the ORX confer odor selectivity. It is the expression profile, but most importantly, the function of these ORCO+ORs (ranging from a few to over a hundred within a single species) that determine a specific olfactory-mediated behavioral event.

For the avoidance of doubt, it should be explained that odorant receptors comprise two polypeptides: the odorant receptor (ORX) and a co-receptor (ORCO). The ORCO is common amongst all odorant receptors, it is the ORX which provides the specificity to the odorant receptor.

Hence an embodiment of the disclosure is wherein the mosquito odorant receptor comprises an odorant receptor (ORX) and a co-receptor (ORCO). More preferably the mosquito odorant receptor is an Aedes aegypti and/or Anopheles gambiae odorant receptor.

The sequences for mosquito odorant receptors is well known in the art. Provided below is a table showing the list of odorant receptors from Aedes aegypti and Anopheles gambiae GenBank accession numbers from which the nucleic acid and subsequent amino acid sequences can be obtained.

TABLE 1
Anopheles gambiae

| Gene name | Accession number | Gene name | Accession number |
|---|---|---|---|
| AgamOrco | XP_312379 | AgamOr50 | XP_310090 |
| AgamOr1 | XP_318674 | AgamOr51 | XP_310087 |
| AgamOr10 | XP_310172 | AgamOr52 | XP_310903 |
| AgamOr11 | XP_320874 | AgamOr53 | XP_310060 |
| AgamOr13 | XP_310070 | AgamOr54 | XP_320702 |
| AgamOr14 | XP_310085 | AgamOr55 | XP_310072 |
| AgamOr15 | XP_310073 | AgamOr56 | XP_313638 |
| AgamOr16 | XP_310066 | AgamOr57 | XP_313640 |
| AgamOr17 | XP_310068 | AgamOr58 | XP_309621 |
| AgamOr18 | XP_310088 | AgamOr59 | XP_311894 |
| AgamOr2 | XP_310173 | AgamOr6 | XP_316227 |
| AgamOr21 | XP_310092 | AgamOr60 | XP_320552 |
| AgamOr22 | XP_317343 | AgamOr61 | XP_320541 |
| AgamOr23 | XP_317710 | AgamOr62 | XP_320553 |
| AgamOr26 | XP_313636 | AgamOr63 | XP_320543 |
| AgamOr27 | XP_313637 | AgamOr64 | XP_320542 |
| AgamOr28 | XP_312203 | AgamOr65 | XP_319640 |
| AgamOr29 | XP_319861 | AgamOr66 | XP_319538 |
| AgamOr3 | XP_317837 | AgamOr68 | XP_318760 |
| AgamOr30 | XP_310061 | AgamOr69 | XP_318761 |
| AgamOr31 | XP_315072 | AgamOr70 | XP_318763 |
| AgamOr33 | XP_001688726 | AgamOr71 | XP_318764 |
| AgamOr34 | XP_320910 | AgamOr72 | XP_318786 |
| AgamOr36 | XP_309205 | AgamOr73 | XP_318789 |
| AgamOr37 | XP_320909 | AgamOr74 | XP_318795 |
| AgamOr38 | XP_312288 | AgamOr75 | XP_321006 |
| AgamOr39 | XP_312289 | AgamOr76 | XP_321005 |
| AgamOr4 | XP_317838 | AgamOr77 | XP_321007 |
| AgamOr41 | XP_310907 | AgamOr8 | XP_321153 |
| AgamOr42 | XP_313200 | AgamOr80 | XP_556129 |
| AgamOr43 | XP_314477 | AgamOr9 | XP_317124 |
| AgamOr44 | XP_314478 | | |
| AgamOr45 | XP_311816 | | |
| AgamOr46 | XP_310062 | | |
| AgamOr47 | XP_310064 | | |
| AgamOr48 | XP_316697 | | |
| AgamOr49 | XP_316698 | | |
| AgamOr5 | XP_317840 | | |

TABLE 2
Aedes aegypti

| Gene name | Accession number | Gene name | Accession number |
|---|---|---|---|
| AaOrco | NM_001358471 | | |
| AaOr2 | NM_001358323 | | |
| AaOr4 | AAEL015147 | | |
| AaOr3 | AAEL017138 | AaOr52 | XM_001663645.2 |
| AaOr4 | XM_001650742.2 | AaOr54 | XM_001651323.2 |
| AaOr6 | XM_021843862.1 | AaOr55 | XM_001654500.3 |
| AaOr8 | XM_001662305 | AaOr57 | XM_021838728.1 |
| AaOr9 | XM_001651704.2 | AaOr58 | XM_001657514.2 |
| AaOr10 | XM_001651706.3 | AaOr59 | XM_001652995.3 |
| AaOr11 | NM 001358172 | AaOr60 | AAEL017041 |
| AaOr13 | XM_001653156.2 | AaOr61 | AAEL017277 |
| AaOr14 | XM_001659191.3 | AaOr62 | AAEL011796 |
| AaOr15 | XM_001659190.3 | AaOr63 | XM_001649148.2 |
| AaOr16 | XM_021855743.1 | AaOr64 | XM_001649149.2 |
| AaOr18 | AAEL017510 | AaOr66 | XM_011495452.2 |
| AaOr19 | AAEL017017 | AaOr67 | AAEL017260 |
| AaOr20 | XM_021851539.1 | AaOr68 | AAEL018076 |
| AaOr21 | XM_011494961.2 | AaOr69 | XM_001658243.3 |
| AaOr22 | XM_021855870.1 | AaOr70 | XM_021845789.1 |
| AaOr23 | XM_021853572.1 | AaOr71 | XM_011495106.2 |
| AaOr24 | XM_011494962.2 | AaOr72 | XM_011495301.2 |
| AaOr25 | XM_001657035.3 | AaOr73 | AAEL006192 |
| AaOr26 | XM_001654502.3 | AaOr74 | AAEL006195 |
| AaOr27 | XM_001654501.3 | AaOr75 | AAEL013419 |
| AaOr28 | XM_021854515.1 | AaOr76 | XM_001663564.2 |
| AaOr29 | XM_021848008.1 | AaOr77 | XM_001663563.2 |
| AaOr30 | XM_001654505.2 | AaOr78 | XM_001663562.2 |
| AaOr31 | XM_001663350.2 | AaOr79 | XM_001663561.2 |
| AaOr32 | XM_021837775.1 | AaOr80 | XM_011494809.2 |
| AaOr33 | XM_011494756.2 | AaOr81 | XM_011494811.2 |
| AaOr34 | XM_021853348.1 | AaOr82 | XM_021840905.1 |
| AaOr36 | XM_011494691.2 | AaOr84 | XM_021851567.1 |
| AaOr37 | XM_021853347.1 | AaOr85 | XM_001648637.3 |
| AaOr38 | XM_021853479.1 | AaOr86 | XM_011495454.2 |
| AaOr40 | XM_001651379.3 | AaOr87 | XM_021853604.1 |
| AaOr41 | XM_001662935.3 | AaOr88 | XM_001648139.3 |
| AaOr42 | XM_021847325.1 | AaOr89 | AAEL017125 |
| AaOr43 | XM_011495034.2 | AaOr90 | AAEL017037 |
| AaOr44 | XM_001657795.3 | AaOr91 | XM_011495085.2 |
| AaOr45 | XM_001649150.3 | AaOr92 | AAEL017065 |
| AaOr47 | XM_011495247.2 | AaOr93 | XM_011495086.2 |
| AaOr48 | XM_021848026.1 | AaOr94 | XM_011494976.2 |
| AaOr49 | AAEL001303 | AaOr95 | XM_011494934.2 |
| AaOr50 | XM_001654504.2 | AaOr96 | XM_011494935.2 |
| AaOr51 | XM_021837777.1 | | |
| AaOr97 | XM_011495296.2 | AaOr114 | XM_021853739.1 |
| AaOr99 | XM_021838590.1 | AaOr115 | XM_021851747.1 |
| AaOr100 | XM_001661597.2 | AaOr116 | XM_021841562.1 |
| AaOr101 | XM_011495166.2 | AaOr117 | XM_011494796.2 |
| AaOr102 | XM_021852918.1 | AaOr118 | XM_021851959.1 |
| AaOr103 | XM_011494932.2 | AaOr119 | XM_011495008.2 |
| AaOr104 | XM_011495347.2 | AaOr120 | AAEL018087 |
| AaOr105 | XM_021846359.1 | AaOr121 | XM_011495435.2 |
| AaOr106 | AAEL017105 | AaOr122 | XM_001663704.3 |
| AaOr107 | XM_021848119.1 | AaOr123 | XM_011495123.2 |
| AaOr108 | XM_021848120.1 | AaOr125 | XM_001664048.2 |
| AaOr110 | XM_011495468.2 | AaOr127 | AAEL017271 |
| AaOr111 | XM_021849006.1 | AaOr128 | AAEL005680 |
| AaOr112 | XM_021849002.1 | AaOr130 | AAEL017285 |
| AaOr113 | XM_021849003.1 | AaOr131 | NC_035107.1 |

Therefore an embodiment of the disclosure is wherein the receptor (ORX) is selected from the receptors shown in Tables 1 and 2 and the co-receptor (ORCO) is selected from receptors shown in Tables 1 and 2.

A preferred embodiment of the disclosure is wherein the co-receptor (ORCO) is selected from AgamOrco and/or AaORCO and the receptor (ORX) is selected from AaOR8, AaOR2 or AaOR11, AgamOR8 or AgamOR2.

Preferably the cell comprises AgamORCO and AgamOR8 or AgamOR2. Preferably the cell comprises AaORCO with AaOR8, AaOR2 or AaOR11.

An example of the amino acid sequence of AgamORCO is shown in SEQ ID NO:1 and AaORCO is shown in SEQ ID NO:2.

In addition to the genes listed in Tables 1 and 2, an embodiment of the disclosure is a mammalian cells comprising a functional fragment, variant, derivative or analogue, of the arthropod chemosensory receptor, or a receptor that is substantially identical to the receptors encoded by the genes listed in Tables 1 and 2

The phrases and terms “functional fragment, variant, derivative or analogue” and the like, as well as their forms used in relation to an arthropod chemosensory receptor, describe a receptor molecule possessing the qualitative biological activity inherent in the arthropod chemosensory receptor. For example, a functional fragment or analog of an arthropod chemosensory receptor that can still respond to the ligand stimuli that receptor. Methods of measuring the binding of a receptor to a ligand are well known in the art, and some methods are disclosed herein below in the Examples section of this document.

The term “substantially identical” to an receptor polypeptide sequence can be interpreted as an identity of at least 70%, 80%, 90%, 95% or more of the receptor sequence and the associated polypeptide sequence. The same term in relation to a nucleic acid sequence can be interpreted as a nucleotide sequence showing an identity of at least 85%, 90%, 95%, 97% or more of the sequence with respect to the compared nucleic acid sequence.

In some embodiments, the mammalian cells comprising an arthropod chemosensory receptor are isolated mammalian cells. In some embodiments, the mammalian cells comprising an arthropod chemosensory receptor are contained in an assay. In some embodiments, the mammalian cells comprising an arthropod chemosensory receptor are adhered to a substrate.

### Screening Methods

A further aspect of the disclosure provides a method of identifying an arthropod, preferably insect, control agent, the method comprising: (a) contacting one or more mammalian cells comprising an arthropod chemosensory receptor (according to any of the embodiments set forth above) with a test compound; and (b) measuring the arthropod chemosensory receptor activity, wherein a change in the arthropod chemosensory receptor activity indicates that the test compound is an arthropod, preferably insect, control agent. An embodiment of this aspect of the disclosure is wherein the method further comprises selecting the test compound as a compound that is an arthropod, preferably insect, control agent.

By “change in the arthropod chemosensory receptor activity” we mean that the receptor has a change in activity in comparison to the receptor not exposed to the test compound.

Where the method further comprises selecting the test compound as a compound that is an arthropod, preferably insect, control agent, then preferably the test compound changes the chemosensory receptor activity by 1.5×, 2×, 2.5×, 3×, 5×, 10×, 20×, 50×or more in comparison to the receptor not exposed to the test compound.

This aspect of the disclosure provides a screening method, for example, a high throughput screening method, comprising the mammalian cell of the first aspect of the disclosure.

This aspect of the disclosure provides a screening method, for example, a high throughput screening method, comprising the mammalian cell of the first aspect of the disclosure.

In such a method, the effect of a test compound on an arthropod chemosensory receptor activity is measured. A change in arthropod chemosensory receptor activity by a test compound indicates modulation of the arthropod chemosensory receptor by the test compound, thereby identifying it as a arthropod chemosensory receptor modulator. Test compounds which modulate arthropod chemosensory receptor activity can be, for example, agonists, antagonists, allosteric modulators, or the like, of the receptor, and are called arthropod chemosensory receptor modulators.

The effect of modulating arthropod chemosensory receptor activity can vary. For example, a modulator may change the behavior of the arthropod in such a way to for it to be attracted to the source of the modulator. In this situation the arthropod chemosensory receptor modulator is an attractant. In another situation the modulator may change the behavior of the arthropod in such a way to for it to be repelled to the source of the modulator. In this situation the arthropod chemosensory receptor modulator is a repellant.

The foregoing embodiments involve measuring the response of an arthropod chemosensory receptor to a test compound. This measuring can be carried out by any suitable means. For example, in some embodiments, the arthropod chemosensory receptor is expressed on the surface of a cell, and compositions comprising the test compound are screened against the cells expressing a chemoreceptor protein in a standard cellular assay.

Various functional assays can be used to determine the activity of the arthropod chemosensory receptor, including assays that measure changes in intracellular calcium cation concentration (for example, Fluorometric Imaging Plate Reader-based Ca2⁺mobilization assay, FLIPR), pERK1/2 activation (for example, high content imaging, HCI), and receptor internalization (for example, TRANSFLUOR). Other assays include measuring the change in membrane voltage using membrane potential dyes, other assays include direct measurement of ion flux through arthropod chemosensory receptor channels using electrophysiological methods, or sodium and calcium imaging. Additional assays include use of a reporter gene assay (for example Luciferase assay) where changes in chemoreceptor function are coupled to changes in the expression or function of another “reporter” protein such to alert the operator that a test compound has modulated the function of the chemoreceptor. These methods are well known in the art and can be readily used by the skilled person in the performance of the method of the disclosure.

The contacting described above can be carried out in any suitable way. For example, in some non-limiting instances, when contacting the test compound to a cell comprising an arthropod chemosensory receptor, the introducing can be carried out in a cell-based assay. Such cell-based assays are well known in the art.

### Uses, Methods, and Ingestible Compositions

A compound identified from the screening method of the disclosure has use as an arthropod control agent.

The expression “control”, “arthropod control”, “insect control” or “arachnid control” or the like has the normal meaning for a skilled person in the technical field.

“Controlling” in the context of the present disclosure defines the ability of an arthropod controlling composition according to the present disclosure to attract, deter, kill or repel an arthropod, preferably deter or repel an arthropod and even more preferably repel an arthropod

“Attracting” according to the present disclosure defines the ability of an arthropod attractant composition according to the disclosure to increase or encourage contact or the presence of an arthropod at the arthropod attractant source, such as in the air, on the surface of an article or on the surface of a vertebrate, such as a human subject or other mammal, preferably an article such as a trapping device, the arthropod attractant compound or composition has been applied to.

“Repellency” according to the present disclosure defines the ability of an arthropod repellent composition according to the present disclosure to minimize, reduce, discourage or prevent approach or the presence of an arthropod at the arthropod repellent source, such as in the air, on the surface of an article or on the surface of a vertebrate, such as a human subject or other mammal, preferably human subject, to which the arthropod repellent compound or composition has been applied to.

“Deterring” according to the present disclosure defines the ability of an arthropod deterrent composition according to the disclosure to minimize, reduce, discourage or prevent contact or the presence of an arthropod at the arthropod deterrent source, such as in the air, on the surface of an article or on the surface of a vertebrate, such as a human subject or other mammal, preferably human subject, to which the arthropod deterrent compound or composition has been applied to. Typically, the deterrent effect is shown when used as feeding deterrent hindering a pest from subsequent food intake or oviposition or physical contact after an initial tasting of the arthropod deterrent compound or composition.

“Spatial Repellency” according to the present disclosure defines the ability of an arthropod repellent composition according to the present disclosure to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to. Typically, the spatial repellency effect is shown when spatial repellent compound or composition released, sprayed, spread or diffused in the air or liquid hinder a pest from entering the zone in which the spatial repellent compound or composition is present. Repellence occurs therefore from a distance, the pest not necessarily entering in direct contact with the treated article or organism to protect.

“Killing” according to the present disclosure defines the ability of arthropod killing composition according to the present disclosure to kill an arthropod at the arthropod killing source, such as in the air, on the surface of an article or on the surface of a vertebrate, such as a human subject or other mammal, preferably human subject, to which the arthropod killing compound or composition has been applied to. When an arthropod killing composition is applied to a plant, an animal or human subject, it is applied in an amount which is killing to the arthropod but not to the subject.

In a particular embodiment, the arthropod control composition is an arthropod repelling composition, preferably an insect repelling composition, more preferably a mosquito repelling composition.

In a particular embodiment, the arthropod controlling source is the surface and/or the air in the vicinity of an article, preferably a candle, coil, an air care product, preferably an electric diffuser, wristband, patch, collar, ear tag, clothes, fabrics, papers, biochar, cardboard, cellulosic pads, bed nets, screen, curtains, furniture, walls, ground or paint, or the surface of a subject, preferably the surface of a vertebrate, such as a human subject or other mammal, preferably human subject, i.e. the skin of a human subject treated with a product such as spray, aerosol, cream, roll on, wristband, lotion, soap, shampoo, sunscreen or patch or a cloth treated with a product such as laundry powder, liquid detergent, spray, lotion, powder.

The arthropod controlling effect according to the present disclosure is determined on mosquitoes using an adapted Warm Body assay as defined in Kröber T, Kessler S, Frei J, Bourquin M, Guerin P M. An in vitro assay for testing mosquito controlling compounds employing a warm body and carbon dioxide as a behavioral activator. J Am Mosq Control Assoc. 2010; 26:381-386. Further information is provided in the accompanying examples The controlling effect, repellence & spatial repellence, according to the present disclosure is determined by testing the Warm Body assay against the yellow fever mosquito, Aedes aegypti Rockefeller strain. A. aegypti is a model organism for controlling tests and one of the recommended model organisms by the World Health Organization (WHO) as it is a very aggressive, anthropophilic mosquito species that shows generally low sensitivity to arthropod controlling compounds. Observations of controlling efficacy were made on host-seeking females of uniform age, 5 to 10 days old selected as mentioned in the publication mentioned hereinabove. Tested hungry females had access to 10% sugar solution but were not blood-fed. Further information is provided in the accompanying examples.

The published protocol has been adapted in not manually counting the landing mosquitoes but automatically using an automatic counting software, the switch from Anopheles gambiae to A. aegypti led to a decrease of mosquitoes' number placed in the tested cage due to the size difference (i.e. 30 mosquitoes instead of 50) and to an increase of lighting as A. aegypti is a diurnal mosquitoes (i.e. 150 lux instead of 4 lux). Further information is provided in the accompanying examples.

The controlling effect, repellence & spatial repellence, according to the present disclosure is also determined according to an arm in the box method adapted from the WHO Guidelines for efficacy testing of mosquito repellents for human skin (WHO/CDS/NTD/WHOPES/2009.4). The readiness of 100 hungry female mosquitoes A. aegypti to a test substance is assessed by comparing the results of an untreated arm to a treated in when inserted into the cage (40×40×40 cm) for 30 seconds (negative control) three times. Further information is provided in the accompanying examples.

The activity for substances to repel arachnids such as ticks may be assessed using the protocol of the in-vitro Warm Plate Assay as defined in Krober T, Bourquin M, Guerin P M. 2013. A standardized in vivo and in vitro test method for evaluating tick repellents. Pestic. Biochem. Phys. 107(2):160-168.

In a particular embodiment, the amount and selection of the substance is in a way that it contributes, enhances or improves both, the arthropod control activity and the hedonic character of the composition.

In one embodiment, the arthropod control composition may further comprise an arthropod control co-ingredient. By “arthropod control co-ingredient” is understood an ingredient capable of imparting additional arthropod controlling benefits to the arthropod controlling effect of the composition herein described.

In one embodiment, the substance herein described is capable to modify, enhance or improve the arthropod controlling effect of the arthropod control co-ingredient, e.g. by reducing the amount of the arthropod control co-ingredient within a composition. This can be particularly beneficial in case the arthropod control co-ingredient is harmful to human subjects at a certain dose or in case the arthropod control co-ingredient has negative olfactive properties at a certain dose.

According to a particular embodiment, the combination of the substance herein described and an arthropod control co-ingredient results in a synergistic arthropod controlling effect.

According to a particular embodiment, the combination of substance herein described and an arthropod control co-ingredient results in a modified, pleasant, enhanced or improved olfactory impression of the overall composition in comparison to its single ingredients.

According to one embodiment arthropod control co-ingredient is selected from the group consisting of: N,N-diethyl-3-methylbenzamide (DEET), ethyl butylacetylaminopropionate (IR3535); para-menthan-3,8-diol (PMD); 1-(1-methylpropoxycarbonyl)-2-(2-hydroxaethyl)piperidin(picaridin); Cedarwood oil China, Cedarwood oil Texas, Cedarwood oil Virginia, Cinnamon oil, Citronella oil, Cornmint oil, Cymbopogon winterianus oil fractionated hydrated cyclized, decanoic acid, Eucalyptus citriodora oil Eucalyptus citriodora oil hydrated cyclized, eugenol, Garlic oil, geraniol, Geranium oil, Lavender, Lavandula hybrida oil, Lavandin oil, Lemon oil, Lemongrass oil, Margosa extract, Metofluthrin, mixture of cis-and trans-p-menthane-3,8 diol, N,N-diethyl-meta-toluamide, nonanoic acid, Rosemary oil, Thyme oil, Wintergreen oil, 2,3,4,5-bis(butyl-2-ene)tetrahydrofurfural (MGK Repellent 11), cineole, cinnamaldehyde, citronellal, citronellol, coumarin, dibutyl phthalate, diethyl phthalate, dimethyl anthranilate, dimethyl phthalate, ethyl vanillin, Eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, Lime oil, limonene, linalool, methyl anthranilate, Mint oil, myrcene, Neem oil, sabinene, β-caryophyllene, (1H-indol-2-yl)acetic acid, anethole, Anise oil, Basil oil, Bay oil, camphor, ethyl salicylate, Evergreen oils (pine oil), (1,3,4,5,6,7-hexahydro-1,3-dioxo-2H-isoindol-2-yl)methyl 2,2-dimethyl-3-(2-methylprop-1-enyl)cyclopropanecarboxylate (d-Tetramethrin), 3-Allyl-2-methyl-4-oxocyclopent-2-enyl-2,2-dimethyl-3-(2-methylprop-1-enyl)-cyclopropanecarboxy late (d-Allethrin), α-cyano-3phenoxybenzyl, 3-(2,2-dichlorovinyl)-2,2-dimethylcyclopropanecarboxylate (Cypermethrin), 2-methyl-4-oxo-3-(prop-2-ynyl)cyclopent-2-en-1-yl 2,2-dimethyl-3-(2-methylprop-1-enyl)cyclopropanecarboxylate (Prallethrin), Acetamiprid, Azadirachtin, Bendiocarb, Bifenthrin, boric acid, Chlorpyrifos, Deltamethrin, Diazinon, Dichlorvos, eugenol, Fipronil, Imidacloprid, linalool, Malathion, Maltodextrin, Metofluthrin, Nicotine, Permethrin, Pyrethrins, Pyrethroids, Rotenone, silicium dioxide (Kieselguhr), S-Methoprene, Spinosad (Spinosyn A), Spinosyn D, Tetramethrin, Transfluthrin, 1-(2,6,6-trimethylcyclohex-2-en-1-yl) but-2-en-1-one, 3-butylidene-2-benzofuran-1-one, 4-ethenyl-2-methoxyphenol, Cognac oil green, Labdanum extract (Cistus spp.), 5-pentyloxolan-2-one, chromen-2-one, 3,7-dimethylocta-2,6-dienal, 4-hydroxy-3-methoxybenzaldehyde, 2-methyl-5-prop-1-en-2-ylcyclohex-2-en-1-one, Mentha spicata oil, 6-hexyloxan-2-one, 5-methyl-2-propan-2-ylcyclohexyl] acetate, Nigella damascena oil, 2-phenylethanol, 6-pentyloxan-2-one, (4-methoxyphenyl) methyl acetate, Syzygium aromaticum oil, 3,4,4a,5,6,7,8,8a-octahydrochromen-2-one, 3,7,7-trimethylbicyclo [4.1.0] hept-3-ene, 2-phenylethyl 2-methylpropanoate, methyl 2-(3-oxo-2-pent-2-enylcyclopentyl)acetate, 4-(2-methoxypropan-2-yl)-1-methylcyclohexene, Mentha piperita oil, 2-methoxy-4-[prop-1-enyl]phenol, 2-methyl-3-(4-propan-2-ylphenyl) propanal, (4-methoxyphenyl)methanol and mixtures thereof.

In a particular embodiment, the arthropod control co-ingredient is comprised in an amount of from 0.02 to 80 wt. %, more preferably in an amount of from 0.05 to 70 wt. %, even more preferably in an amount of from 0.1 to 60 wt. %, based on the total weight of the composition. Thereby, it is understood that the composition comprises the arthropod control co-ingredient in a minimum amount of at least 0.02 wt. %, at least 0.05 wt. % or at least 0.1 wt. % and a maximum amount of not more than 80 wt. %, not more than 70 wt. % or not more than 60 wt. %, based on the total weight of the composition.

In a particular embodiment, within the limitations of the amount of the substance in the composition as stated above, the substance in the composition of the disclosure and the arthropod control co-ingredient are comprised in the composition in a weight range of 90:10 to 10:90, preferably in a weight range of 80:20 to 20:80, more preferably in a weight range of 65:35 to 35:65 and most preferably in a weight range of 60:40 to 40:60. It is herein also understood that substance and the arthropod control co-ingredient can be comprised in the composition in any weight range combination as mentioned herein-before, such as 90:10 to 20:80, preferably 35:65 and more preferably 40:60, 80:20 to 10:90, preferably 35:65 and more preferably 40:60, 65:35 to 10:90, preferably 20:80 and more preferably 40:60 or 40:60 to 10:90, preferably 20:80 and more preferably 35:65.

In one embodiment, the arthropod control composition may further comprise perfume ingredients. Perfume ingredients are understood as contributing, modifying, enhancing or improving the olfactory character of the composition but do not contribute to, enhance or improve the arthropod controlling effect of the composition. Perfume ingredients providing such hedonic effects and suitable for use in the composition of the disclosure are known in the art and can be readily identified by the skilled person.

The arthropod control composition can further comprise a carrier. By “carrier” is understood a material with which the active compound is mixed or formulated to facilitate its application a locus or other object to be treated, or its storage, transport and/or handling. Said carrier may be of inorganic or organic or of synthetic natural origin. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly cannot be exhaustive. However, one can cite as non-limiting examples, solvents such as butylene or propylene glycol, glycerol, dipropylene glycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethanol, tri-ethyl citrate, 2-methylprop-1-ene and 2-(2-ethoxyethoxy)ethanol or mixtures thereof, particular suitable are dipropylene glycol, 2-methylprop-1-ene and 2-(2-ethoxyethoxy)ethanol and mixtures thereof.

For the compositions which comprise both a carrier, other suitable carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company) like DowanoI™ DPMA (Glycol Ether Acetate), or Augeo® Clean Multi (isopropylidene glycerol; origin: Solvay), or hydrogenated castors oils such as those known under the trademark Cremophor® RH 40 (origin: BASF).

Solid carrier is meant to designate a material to which the arthropod control composition or some element of the arthropod control composition can be chemically or physically bound. In general, such solid carriers are employed either to stabilize the composition, or to control the rate of evaporation of the compositions or of some ingredients. The use of solid carrier is of current use in the art and a person skilled in the art knows how to reach the desired effect. However, by way of non-limiting example of solid carriers, one may cite absorbing gums or polymers or inorganic material, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di-or trisaccharides, natural or modified starches, hydrocolloids, cellulose derivatives, polyvinyl acetates, polyvinylalcohols, proteins or pectins, or other such materials. The encapsulation is a well-known process to a person skilled in the art, and may be performed, for instance, by using techniques such as spray-drying, agglomeration or yet extrusion; or consists of a coating encapsulation, including coacervation and complex coacervation technique.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively, one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate.

Many articles have been published relating to the encapsulation by polycondensation of amino resins, namely melamine-based resins with aldehydes. Such articles already describe the various parameters affecting the preparation of such core-shell microcapsules. These documents are known to the skilled person and the contents therein be used in the field of the present disclosure.

The present disclosure also relates to a method for arthropod, preferably insect, control which comprises bringing an arthropod, preferably insect, into direct contact or in contact with vapors of a composition as described hereinabove.

For the sake of clarity, the arthropod controlling composition according to the present disclosure can be applied to the air, to the surface of an article, the air in the vicinity of the surface of an article or the surface of a subject by usual methods known in the art such as spraying, applying, wearing or diffusing.

In a particular embodiment, the arthropod controlling composition according to the present disclosure is applied to the surface of an article, the air in the vicinity of the surface of an article or to the surface of an animal or subject.

In a particular embodiment, the article can be an arthropod control article as described hereinbelow and in particular, can be a candle, coil, an air care product, preferably an electric diffuser, wristband, patch, collar, ear tag, clothes, fabrics, papers, biochar, cardboard, cellulosic pads, bed nets, screen, curtains, furniture, paint, walls, ground, spray, aerosol, cream, roll on, wristband, lotion, soap, shampoo, sunscreen, laundry powder, liquid detergent, spray, lotion, powder.

In a particular embodiment, the surface of a subject is the surface of a human or animal subject, preferably the surface is a human subject, i.e. the skin of a human subject.

The present disclosure also relates to a use of a composition as defined hereinabove to control arthropods, preferably insects.

The present disclosure also relates to an arthropod control article comprising the arthropod control composition as described hereinabove.

By “arthropod control article” is understood to designate a consumer product which delivers at least an arthropod controlling effect to the surface or space to which it is applied (e.g. skin, hair, textile, or home surface). In other words, an arthropod controlling article according to the disclosure is a consumer product which comprises a functional formulation, as well as optionally additional benefit agents, corresponding to the desired consumer product, and an arthropod controlling amount of at least one of the substances. For the sake of clarity, said consumer product is a non-edible product.

The nature and type of the constituents of the consumer product do not warrant a more detailed description here, which in any case would not be exhaustive, the skilled person being able to select them on the basis of his general knowledge and according to the nature and the desired effect of said product.

Non-limiting examples of suitable consumer products include a perfume, such as a fine perfume, a splash or eau de parfum, a cologne or a shave or after-shave lotion or a cream or gel; a fabric care product, such as a liquid or solid detergent, a laundry powder, a fabric softener, a liquid or solid scent booster, a fabric refresher, an ironing water, a paper, a bleach, a carpet cleaner, a curtain-care product; a body-care product, such as a hair care product (e.g. a shampoo, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e a floor, bath, sanitary or a window-cleaning) detergent; a leather care product; a car care product, such as a polish, a wax or a plastic cleaner; a candle;

a spray, a coil, an air care product, a piezo diffuser, a liquid electric diffuser, a diffusor, a rubber septum, a wristband, a patch, a collar, an ear tag, clothes, fabrics, papers, a biochar, a cardboard, cellulosic pads, bed nets, a screen, curtains, a varnish or a paint, more preferably a candle, a spray, a coil, an electric diffuser, a piezo diffuser, a liquid electric diffuser, a diffusor, a rubber septum, a wristband, a patch, a collar, an ear tag, clothes, fabrics, papers, a biochar, a cardboard, cellulosic pads, bed nets, a screen, curtains, a varnish or a paint.

In a preferred embodiment of the disclosure, the consumer product is an air care product, preferably an electric diffuser. In this embodiment of the disclosure, the substance in the arthropod, preferably insect, control composition is present at certain quantities.

Some of the above-mentioned consumer products may represent an aggressive medium for the members of the substances, so that it may be necessary to protect the latter from premature decomposition, for example by encapsulation or by chemically binding it to another chemical which is suitable to release the disclosure's ingredient upon a suitable external stimulus, such as an enzyme, light, heat or a change of pH.

### EXAMPLES

### Introduction

We demonstrate a reliable system for the expression, function and high-throughput screening of mosquito odorant receptor targets after transduction of U2OS cells with recombinant viruses. Specifically, the use of U2OS host cell line results in an assay windows that are even greater than signals detected in HEK293 cells either transiently or stably expressing the mosquito odorant receptors. This novel use of the U2OS expression platform was used for the HTS and discovery of novel modulators of several mosquito olfactory receptors.

### Materials and Methods

Baculovirus construction and amplification. To initiate Bacmam virus generation the polyhedrin promoter in the pFastBac vector of the Bac-to-Bac Baculovirus expression system (ThermoFisher) was replaced with the mammalian CMV promoter to generate pBacmam1, an expression vector which allows for gene expression in mammalian cells. The cDNAs encoding mosquito odorant receptors were cloned into a modified pBacmam1 and virus was subsequently produced according to the protocols outlined in the Bac-to-Bac system using Spodoptera frugiperda (sf9) cells. Virus titers were determined by viral plaque assay. Large-scale virus stocks with titers of ˜7×10⁷ pfu/ml were stored at 4° C.

Cell culture and transduction. U2OS and HEK293 cells were grown on T150 flasks with Dulbecco's modified eagle medium supplemented with 10% fetal bovine serum and 1% Penicillin/Streptomycin in a 37° C. incubator under a 5% CO₂ atmosphere. For transductions, cell cultures were pooled at 1.5×10⁶ cell/ml, centrifuged at 1000 xg for 5 min, and resuspended in fresh medium. Resuspended cultures were inoculated at a multiplicity of 1-20 pfu/cell with baculovirus encoding AaORCO or AaORCO with AaOR8, AaOR2 or AaOR11 and AgORCO with AgOR8 or AgOR2. A further untransduced culture was used as a control. Cultures were maintained in an incubator at 37° C. and used for electrophysiology or FLIPR measurements 1-4 days after infection.

FLIPR. One day prior to conducting the assay, U2OS and HEK293 cells were transduced with the corresponding OR using the bacmam method described above and seeded into 384-well clear-bottom plates at ˜20,000 cells per well (80 μl per well) using a multi-channel dispenser. The cell plates were incubated in a 5% CO₂ humidified incubator at 37° C. for 24 hrs. On the day of the assay, the plates were washed with Phosphate Buffer Saline (PBS) using a plate washer (Bio-Tek Instruments, Winooski, VT). Cells were incubated in either a fluorescent Ca²⁺dye (Fluo-4-AM) or Membrane Potential dye solution for 1 hr at room temperature. Agonist-dependent fluorescence measurements were performed on the cell plate using a FLIPRTETRA® (Molecular Devices). The illumination wavelength of the FLIPR light-emitting diode module using the Ca²⁺dye was 470-495 nm, and the emissions were recorded at 515-575 nm. The illumination wavelength of the FLIPR light-emitting diode module using the membrane potential dye was 510-545 nm, and the emissions were recorded at 565-625 nm. Baseline recordings were acquired for 7 seconds before the addition of the agonist. Agonist-induced increases in fluorescence were acquired for 103 s with 1-3 s intervals (63 data points). Data is quantitated as delta F=Fₘₐₓ−Fₘᵢₙ, where Fₘₐₓ was the maximum response in arbitrary fluorescence units, and Fₘᵢₙ was the baseline arbitrary fluoresce unit value. FLIPR data analysis was performed with ScreenWorks (Molecular Devices).

Planar Patch Clamp Electrophysiology Assay. One day prior to conducting the assay, U2OS cells were transduced using 10 pfu/cell with the corresponding OR using the bacmam method described above in T175 flasks and incubated in a 5% CO₂ humidified incubator at 37° C. for 24 hrs. On the day of the assay, the cells were lifted, washed once in PBS and loaded on a Nanion Synchropatch384 instrument and currents were recorded using voltage clamp in standard whole-cell configuration at-90 mV on an 8-hole version chip (NPC-384T).

Warm Body Assay. The rationale of the WBA is to attract female mosquitoes to an electrical device that is heated mimicking the human body temperature. Briefly, a glass Petri dish is attached to the outer surface of the warm body, assuring a temperature of 34±0.5° C. on the surface of the Petri dish. Attraction is measured by monitoring the number of mosquito landings on the petri dish using a camera equipment installed outside of the cage. To screen *potential repellent stimuli, the interior of the glass Petri dish is treated with ethanol as a control or with the test stimulus diluted in ethanol and the number of mosquito landings on the Petri dish is filmed by the video camera for 2 minutes.

### Results & Figures

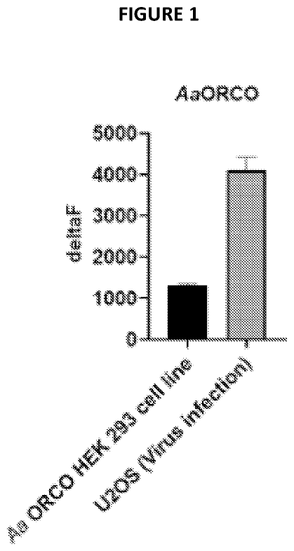

FIG. 1 shows U2OS cells infected with the odorant receptor co-receptor AaORCO provided a greater fluorescence assay window than HEK293 cell stably expressing the same gene. Fluorescence responses observed in a FLIPR system in cells stably expressing AaORCO compared to U2OS cells infected with the same gene. Cells were loaded with a fluorescent indicator and challenged with AaORCO activator VUAA1. U2OS cells are thus better suited for mosquito OR HTS assays than a HEK293 stable cell line by providing greater signal intensity and signal-to-noise ratios.

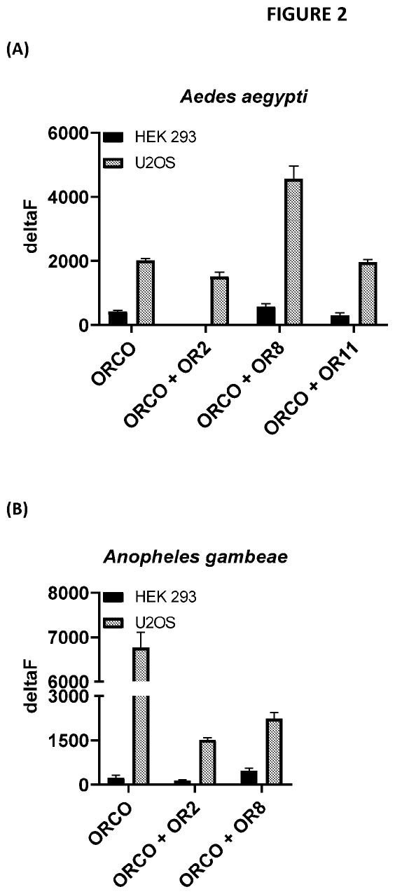

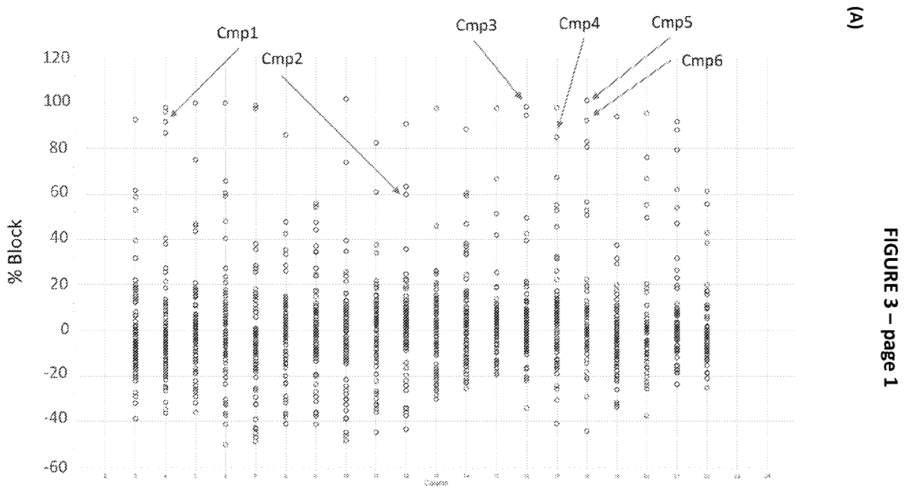

FIG. 2 shows A greater fluorescence assay window was observed in infected U2OS compared to infected HEK293 cells regardless of mosquito odorant receptor target or species expressed. Fluorescence responses observed in a FLIPR system in HEK293 and U2OS cells infected with odorant receptors from two different mosquito species, Aedes (A) and Anopheles (B). Cells were loaded with a fluorescent indicator and challenged with the corresponding OR activator. U2OS cells are particularly efficient at providing a better fluorescence signal for mosquito OR overall making them amenable to HTS screening for the discovery of novel repellants and attractants for several mosquitoes species and targets.

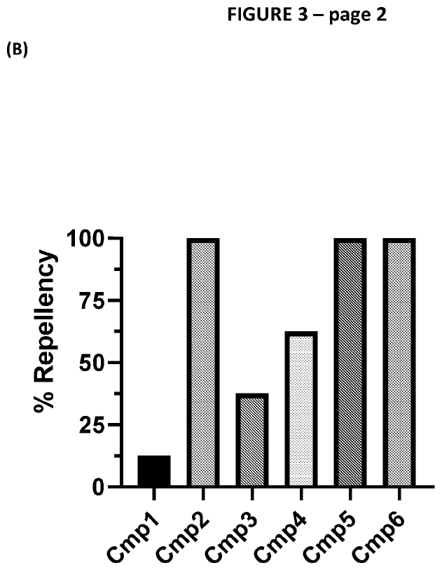

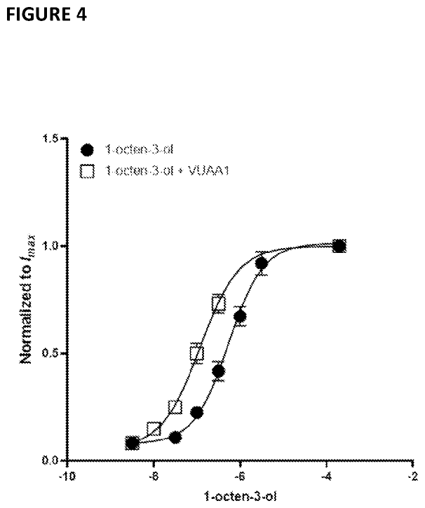

FIG. 3. A, a scatter dot plot representation of 1400 compounds screened for antagonist activity on the Aa odorant receptor AaOrco/Or8. The physiological ligand, 1-octen-3-ol, for AaOrco/Or8 was used in the screening. B, examples of compounds with more than 50% antagonist activity in the AaOrco/Or8 screen show mosquito repellency (scale from 0-100, 0=no repellency and 100=high repellency).

### Conclusion

Expressing mosquito odorant receptors in U2OS cells results in greater functional activity compared to other common expression strategies and cells such as HEK293 cells. Do to this enhanced activity, the use of U2OS cells are thus better suited for high-throughput screening assays than HEK293 cells by providing greater signal intensity and signal-to-noise ratios. To overcome poor expression of receptors in HEK293 cells it is common in the field to mutate the gene sequence or attach an additional protein tag to facilitate surface trafficking and function. The current disclosure has the important added advantage of being able to express, study and screen the wild type or native mosquito odorant receptor protein as it exists in nature. Mosquito odorant receptor modulators identified using the native odorant receptors have a higher likelihood of working in real-world applications. An additional advantage to using U2OS cells is that they are particularly efficient at being transduced by the Bacmam virus thus allowing a fast and convenient way to combine and express many mosquito OR subunit without the need to generate and maintain potentially hundreds of individual stable cell lines.

1. A mammalian cell comprising an arthropod chemosensory receptor.

2. The cell of claim 1 wherein the cell is from an immortalized cell line.

3. The cell of claim 2 wherein the immortalized cell line is not an embryo-derived cell line.

4. The cell of any of the previous claims wherein the cell is a U2OS cell.

5. The cell of any of the previous claims wherein the receptor is transiently expressed in the mammalian cell.

6. The cell of claim 5 wherein the receptor is transiently expressed using a baculovirus expression vector system.

7. The cell of any of the previous claims wherein the chemosensory receptor is an olfactory receptor.

8. The cell of any of the previous claims wherein the arthropod is an insect and the chemosensory receptor is an insect odorant receptor.

9. The cell of any of the previous claims wherein the receptor is a mosquito odorant receptor.

10. The cell of claim 9 wherein the mosquito odorant receptor comprises an odorant receptor (ORX) and a co-receptor (ORCO).

11. The cell of claim 10 wherein the mosquito odorant receptor is anAedes aegypti and/or Anopheles gambiae odorant receptor.

12. The cell of claim 11 wherein the co-receptor (ORCO) is selected from AgamOrco and/or AaORCO and the receptor (ORX) is selected from AaOR8, AaOR2 or AaOR11, AgamOR8 or AgamOR2.

13. The cell of any one of the previous claims wherein the cell is contained in a cell-based assay or disposed on a solid support.

14. A method of identifying an arthropod control agent, the method comprising:
(a) contacting one or more mammalian cell comprising an arthropod chemosensory receptor according to any one of the previous claims with a test compound; and
(b) measuring a receptor activity,
wherein a change in the receptor indicates that the test compound is an arthropod control agent modulator.

15. The method of claim 14 wherein modulation of the receptor activity indicates that the test compound is an arthropod control agent.

16. Use of an identified compound of claim 14 or a selected compound of claim 15 as an arthropod repellant.

17. An arthropod, preferably insect, control article comprising an arthropod, preferably insect, control composition as defined in claim 15 or 16.
